(19) United States
(12) Patent Application Publication
(10) Pub. No.: US 2012/0177757 A1
(43) Pub. Date: Jul. 12, 2012

(54) Natural Preparation for Treatment of Skin Sores and Lesions

(76) Inventors: Adel Jamil Hidmi, Jerusalem (IL); Murad Mahmud Amdalla, Ein Naquba (IL); Thaer Yousef Hantash, Ramallah (IL)

(21) Appl. No.: 13/344,229

(22) Filed: Jan. 5, 2012

(30) Foreign Application Priority Data
Jan. 12, 2011 (IL) 210587

(51) Int. Cl. A61K 36/537; A61P 17/02; A61K 36/48; A61K 36/534; A61K 36/8962

(52) U.S. Cl. 424/746; 424/747; 424/754; 424/757

(57) ABSTRACT

The present invention refers to a preparation made of natural ingredients which contains soybean oil, sesame oil, and curcumin powder.

That is intended for the treatment of general skin sores and lesions, particularly for chronic wounds those associated with diabetes (diabetic foot ulcers), also in patients in their older age or during steroid treatment whilst the normal wound healing ability is seriously weakened.

### TECHNICAL FIELD

The present invention refers to a preparation that contains natural ingredients and is intended mainly for the treatment of general skin sores and lesions, particularly chronic wounds that take a long period of time to heal which associated with many diseases mainly diabetes. So it is a valuable pharmaceutical mean to the treatment of diabetic foot ulcers, neuropathic ulcers, and diabetic venous ulcers.

### BACKGROUND ART

Diabetes is one of the main health problems that influence the life of human being for centuries, it is widely recognized as one of the leading causes of death and disability in the world.

Diabetes is associated with long-term serious complications that affect almost every part of the body. Among other things, diabetes leads to constriction of blood vessels (vasoconstriction), including numerous tiny blood vessels, in a way that significantly reduces the blood flow to all body parts mainly peripheral parts in limbs, leading in many cases to the formation of sores, skin inflammations, fungal infections, bacterial infections, nerve damage and a variety of other skin sores and lesions and even to amputation.

The known methods for treatment of chronic wounds have some disadvantages and drawbacks, some of them are very expensive, others demand regimens which are very difficult or painful for a patient, and quite frequently they are simply not sufficiently effective since chronic wounds are characterized by a very high recurrence rate.

The preparation, subject of the present invention, is intended to treat skin sores, inflamed skin, skin infected by fungi and/or bacteria and a wide variety of skin lesions caused by diabetes that affect foot, as well as general conditions, such as wounds and cuts, acne, bedsores, cankers, cold sores, skin stretches common among pregnancy, skin rashes (babies, children and adults), burn wounds, skin burns, injuries due to over-exposure of skin to sun, cracked heel, hemorrhoids, varicose veins and so on. The preparation, subject of the present invention, is also intended for the treatment of wrinkles, it smoothes and rejuvenates the skin. Therefore, it is an objective of the present invention to provide useful pharmaceutical means for the treatment of chronic wounds, preferably diabetic foot ulcers, which would be effective and economically significant preparation.

For the sake of convenience and the flow of description, the word “lesion” will refer in the present patent application to the entire variety of skin sores and lesions mentioned above, and the expression “wrinkle treatment” will refer to the treatment of wrinkles, as well as to the smoothing and rejuvenation of the skin.

### THE INVENTION

The preparation, subject of the present invention, (hereinafter referred to as “the preparation”) is intended for the treatment of lesions and wrinkles. The preparation is based on natural ingredients that can be purchased in ready-to-use condition and do not require any special manufacturing.

The preparation is a liquid (although it can be viscous or have other consistencies such as a cream, powder, etc.) that is applied to the treatment site (lesions or wrinkles). The preparation should be applied several times a day, and after several days of treatment, positive results are obtained.

The preparation, subject of the present invention, is effective and efficient for the treatment of various types of skin lesions and wrinkles. It also restores damaged sensory (a peripheral nerve) cells in the injured area, preventing nerve damage associated mainly with diabetic foot, causes blood vessels to dilate (Vasodilation), leading to better blood flow to the skin and cells in the injured areas and to their restoration. The preparation accelerates the development and rejuvenation of skin cells (division and multiplication of skin cells assist restoration of damaged areas), through the active mechanism against different bacteria, fungi, and microbes that produce severe infections in the injured area mainly the foot.

The basic ingredients of the preparation: The preparation, subject of the present invention, contains the following three basic ingredients: (1) soybean oil; (2) sesame oil; and (3) curcumin powder. The recommended ratio of the above-mentioned ingredients in the preparation is: one portion (weight and/or volume) soybean oil, one portion sesame oil, and three portions curcumin powder.

Method of preparation: The three above-mentioned ingredients are mixed for 36 hour at room temperature and the mixture is filtered (mainly to remove residual curcumin powder that was not dissolved in the sesame and soybean oils). The resulting liquid is the preparation, subject of the present invention.

Additional ingredients: The inventors recommend adding one or more of the following ingredients to the preparation: olive oil, black cumin oil, grape seed oil, Jojoba oil, garlic oil, Glycyrrhiza glabra (licorice) as a powder or aqueous extract or alcoholic extract, capsaicin oil, clove oil, mint oil, salvia (sage) oil, shea butter, pomegranate essence, cardamom oil, cumin oil, caraway oil, anise oil, and thyme oil.

If one (or more) of the above-mentioned ingredients is added, it could be added at a ratio of one portion (equal to the portion size of sesame or soybean oil) or even less or more.

If pomegranate essence is added, the inventor recommends that it be produced in one of the following manners: (a) Dry the pomegranate, boils it in water, filter and the filtrate is concentrated by removing the water; or (b) thy the pomegranate, grind it, and mix powder into the preparation mixture for 36 hour at room temperature and the mixture is filtered. Any solid parts of the ground pomegranate will be filtered out.

The ingredients mentioned in the preparation (oils and/or powders) means not only the ingredient itself as it is usually soled, but also include any component or material in each used oil and/or powder and any type of extraction and preparation for each used component. Therefore the terms “soybean oil” “sesame oil”, “curcumin powder”, “olive oil”, “black cumin oil”, “grape seed oil”, “Jojoba oil”, “garlic oil”, “Glycyrrhiza glabra (licorice)”, “capsaicin oil”, “clove oil”, “mint oil”, “salvia (sage) oil”, “shea butter”, “pomegranate essence”, “cardamom oil”, “cumin oil”, “caraway oil”, “anise oil” and thyme oil” means in this patent application also any component or material in each used oil and/or powder and any type of extraction and preparation for each used component.

What is claimed is:

1. A preparation for the treatment of lesions and/or wrinkles that contains the following ingredients: soybean oil, sesame oil, and curcumin powder.

2. The preparation mentioned in claim No. 1, also containing one or more of the following ingredients: olive oil, black cumin oil, grape seed oil, Jojoba oil, garlic oil, Glycyrrhiza glabra (licorice) as a powder or aqueous extract or alcoholic extract, capsaicin oil, clove oil, mint oil, salvia (sage) oil, Shea butter, essence of pomegranate, cardamom oil, cumin oil, caraway oil, anise oil, and thyme oil.
